(19) United States
(12) Patent Application Publication
(10) Pub. No.: US 2018/0353272 A1
(43) Pub. Date: Dec. 13, 2018

(54) METHOD OF SPRAYING CERAMIC POWDER

(71) Applicant: JAMES D. EMMONS, WOODWAY, TX (US)

(72) Inventor: JAMES D. EMMONS, WOODWAY, TX (US)

(21) Appl. No.: 15/619,697

(22) Filed: Jun. 12, 2017

(51) Int. Cl. A61C 13/083; A61K 6/083; A61K 6/02; A61C 13/00

(52) U.S. Cl. CPC A61C 13/0835; A61C 13/0006; A61K 6/0205; A61K 6/0835

(57) ABSTRACT

A method and system for spraying ceramic powder onto a surface wherein the method eliminates the need for chlorofluorocarbon (CFC)/butane propellants, may include preparing a water soluble suspension polymer mixture; adding the mixture to a bag-on-valve delivery system; and spraying a fine mist coating of the mixture from the bag-on-sale delivery system to the surface.

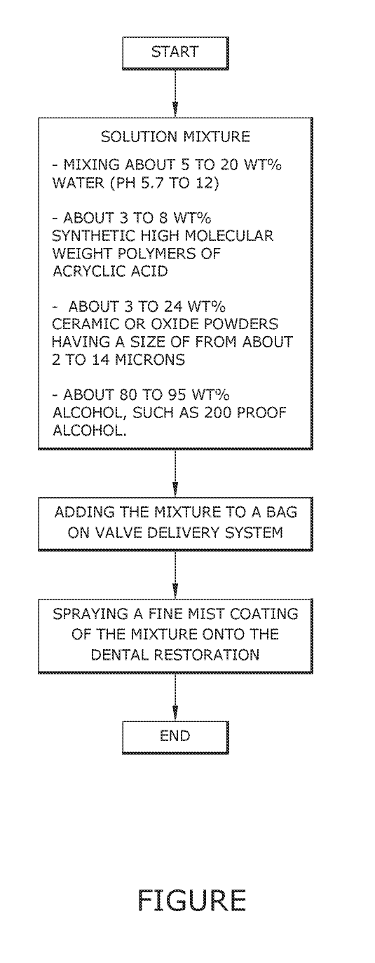

### BACKGROUND

The embodiments herein relate generally to dental restorations, and more particularly, to a method of spraying ceramic powder on dental restorations, wherein the method eliminates the need for chlorofluorocarbon (CFC) or butane propellants.

Conventionally, spraying ceramic powders requires the use of hazardous propellants, such as CFC and butane. These propellants are hazardous to the environment and flammable.

Therefore, what is needed is a method for spraying ceramic powder, wherein the method eliminates the need for CFC/butane propellants.

### SUMMARY

Some embodiments of the present disclosure include a method and system for spraying ceramic powder onto a surface wherein the method eliminates the need for chlorofluorocarbon (CFC)/butane propellants. The method and system may include preparing a water soluble suspension polymer mixture; adding the mixture to a bag-on-valve delivery system; and spraying a fine mist coating of the mixture from the bag-on-sale delivery system to the surface.

### BRIEF DESCRIPTION OF THE FIGURES

The detailed description of some embodiments of the invention is made below with reference to the accompanying FIGURES, wherein like numerals represent corresponding parts of the FIGURES.

FIGURE is a flow chart describing one embodiment of the present disclosure.

### DETAILED DESCRIPTION OF CERTAIN EMBODIMENTS

In the following detailed description of the invention, numerous details, examples, and embodiments of the invention are described. However, it will be clear and apparent to one skilled in the art that the invention is not limited to the embodiments set forth and that the invention can be adapted for any of several applications.

The method of the present disclosure may be used to spray ceramic powder onto a surface, such as a dental restoration, and may comprise the following elements. This list of possible constituent elements is intended to be exemplary only, and it is not intended that this list be used to limit the method of the present application to just these elements. Persons having ordinary skill in the art relevant to the present disclosure may understand there to be equivalent elements that may be substituted within the present disclosure without changing the essential function or operation of the device.

1. Preparing a water soluble suspension polymer mixture

2. Adding the mixture to a bag-on-valve delivery system

3. Spraying the mixture from the bag-on-valve delivery system

The various elements of the method of the present disclosure may be related in the following exemplary fashion. It is not intended to limit the scope or nature of the relationships between the various elements and the following examples are presented as illustrative examples only.

By way of example, and referring to FIGURE, some embodiments of the present disclosure include a method of spraying ceramic powder onto a surface, such as a dental restoration, wherein the method eliminates the need for CFC/butane propellants, the method comprising preparing a water soluble suspension polymer mixture; adding the mixture to a bag-on-valve delivery system; and spraying a fine mist coating of the mixture from the bag-on-sale delivery system to the surface, such as the dental restoration surface.

In embodiments, preparing the water soluble suspension polymer mixture may comprise mixing water; polymers of acrylic acid; ceramic or oxide powders; and alcohol. Suitable polymers of acrylic acid include synthetic high molecular weight polymers of acrylic acid, such as a carbomer, which is a polymer of acrylic acid cross-linked with a polyfunctional compound, like a poly (acrylic acid) or polyacrylate. Suitable ceramic or oxide powders include those having a size of from about 2 to about 14 microns, such as borosilicate glass. Suitable alcohols include 200 proof alcohol, such as SD 40 200 ethanol, which is an official preparation of ethanol and conventionally used as a disinfectant, solvent, and preservative.

The components of the water soluble suspension polymer mixture may be combined in any suitable amounts. For example, some embodiments of the mixture may comprise from about 5 to about 20 wt % water, wherein the water may have a pH of from about 5.7 to about 12; about 3 to about 8 wt % polymers of acrylic acid; about 3 to about 24 wt % powders; and about 80 to about 95 wt % alcohol. Including the ingredients in these amounts provides constant suspension while simultaneously affecting evaporation of the atomized mixture. Specifically, the water soluble polymer mixed with the alcohol and water provide the particles, such as dental glass particles, and oxides to be suspended inside the bag-on-valve in a constant suspension, enabling the atomization of the mixture as the actuator allows the material in the bag-on-valve to be dispensed in a fine, even layer having a thickness of, for example, from about 0.01 to about 0.2 mm. The layer may be applied, for example, onto dental restorations to achieve bonding or glazing/shading of ceramic to dental restorations of metal or ceramic underlayment. The layer may be applied without the use of aerosol propellants or other products requiring hazmat shipping requirements. If the mixture is not maintained as a constant suspension of the powder materials within the mixture, the method may not function properly.

If the water soluble polymer is not included in the mixture, the powder liquid may separate and settle in the middle of the bottom of the bag in the bag-on-valve canister. The grit size range may also be important for allowing the material to flow through the actuator valve without clogging the valve. The alcohol may be necessary to establish the desired evaporation rate. Thus, the ingredients described above may be necessary to make the system function correctly.

Suitable bag-on-valve systems include bag housed within a container, such as a can, wherein the bag is attached to an actuator via a valve, such that when the actuators are pressed or otherwise activated, the material from the bag sprays through the valve, out of the actuator, and onto the desired surface. When the bag-on-valve system is not in use, the water soluble suspension polymer mixture is stored within the bag. Because of the composition of the mixture, the mixture may be maintained as a constant suspension of the powder materials within the mixture without the powder separating and settling in the bottom of the bag.

While the above embodiments describe using the method of the present disclosure for spraying ceramic powder on dental restorations, such as titanium implant abutments, the method of the present disclosure may be used in any desired field, such as the ceramic arts and pottery fields.

Persons of ordinary skill in the art may appreciate that numerous design configurations may be possible to enjoy the functional benefits of the inventive systems. Thus, given the wide variety of configurations and arrangements of embodiments of the present invention the scope of the invention is reflected by the breadth of the claims below rather than narrowed by the embodiments described above.

What is claimed is:

1. A method of spraying ceramic powder onto a surface wherein the method eliminates the need for chlorofluorocarbon (CFC)/butane propellants, the method comprising:
preparing a water soluble suspension polymer mixture;
adding the mixture to a bag-on-valve delivery system; and
spraying a fine mist coating of the mixture from the bag-on-sale delivery system to the surface.

2. The method of claim 1, wherein preparing the water soluble suspension polymer mixture comprises mixing water, polymers of acrylic acid, powders, and alcohol.

3. The method of claim 2, wherein the polymers of acrylic acid include synthetic high molecular weight polymers of acrylic acid.

4. The method of claim 2, wherein the powders are a member selected from the group consisting of ceramic powders and oxide powders.

5. The method of claim 4, wherein the powders have a size of from about 2 to about 14 microns.

6. The method of claim 2, wherein the alcohol comprises 200 proof alcohol.

7. The method of claim 1, wherein the water soluble suspension polymer mixture comprises:
about 5 to about 20 wt % water,
about 3 to about 8 wt % polymers of acrylic acid;
about 3 to about 24 wt % powders; and
about 80 to about 95 wt % alcohol.

8. The method of claim 1, wherein the fine mist coating has a thickness of from about 0.01 to about 0.2 mm.

9. A system for spraying a ceramic powder onto a surface, the system comprising:
a bag-on-valve delivery system; and
a volume of water soluble suspension polymer mixture held within the bag-on-valve delivery system, the water soluble suspension polymer mixture comprising
water;
polymers of acrylic acid;
powders; and
alcohol,
wherein the system eliminates the need for using chlorofluorocarbon (CFC)/butane propellants.

10. The system of claim 9, wherein the water soluble suspension polymer mixture comprises:
about 5 to about 20 wt % water,
about 3 to about 8 wt % polymers of acrylic acid;
about 3 to about 24 wt % powders; and
about 80 to about 95 wt % alcohol.
